SEQUENCE LISTING (1 sequence)

SEQ ID NO: 1 - protein, 406 aa, Homo sapiens [sequence omitted]

(19) United States
(12) Patent Application Publication
(10) Pub. No.: US 2019/0015532 A1
(43) Pub. Date: Jan. 17, 2019

(54) QUANTITATIVE PET IMAGING OF TISSUE FACTOR EXPRESSION USING 18F-LABLED ACTIVE SITE INHIBITED FACTOR VII

(71) Applicant: RIGSHOSPITALET, Copenhagen (DK)

(72) Inventors: Andreas KJAER, Frederiksberg (DK); Carsten HAAGEN NIELSEN, Copenhagen N (DK)

(21) Appl. No.: 16/070,073

(22) PCT Filed: Jan. 13, 2017
(86) PCT No.: PCT/DK2017/050006

§ 371 Date: Jul. 13, 2018

(30) Foreign Application Priority Data
Jan. 15, 2016 (DK) PA 2016 70019

(51) Int. Cl. A61K 51/08

(52) U.S. Cl. CPC A61K 51/088

(57) ABSTRACT

There is provided a positron-emitting 18F-labelled Factor VII for non-invasive PET imaging of tumor TF expression in humans. More specifically the invention relates to human TFPET imaging of pancreatic cancer metastasis for diagnosis, staging, treatment monitoring and especially as an imaging biomarker for predicting prognosis, progression and recurrence.

### FIELD OF THE INVENTION

The present invention relates to a positron-emitting 18-F labelled Factor VII for noninvasive PET imaging of Tissue Factor expressing tumors in humans. More specifically the invention relates to human TF PET imaging of any solid cancer disease for diagnosis, staging, treatment monitoring, companion diagnostics and especially as an imaging biomarker for predicting prognosis, progression and recurrence.

### BACKGROUND OF THE INVENTION

Tissue factor (TF) is a 47 kDa transmembrane protein, which binds factor VII (FVII) with high affinity. The resulting complex initiates the extrinsic coagulation cascade essential for normal hemostasis. Upon binding to TF, the zymogen FVII gets activated to the serine protease, FVIIa; and the TF:FVIIa complex further activates factor X eventually leading to thrombin generation and hemostasis.

In addition to its role in coagulation, TF plays a central role in cancer progression, angiogenesis, invasion and hematogeneous metastatic dissemination. Many tumors express various levels of cells surface TF, and the TF:FVIIa complex has been shown to activate protease activated receptor 2 (PAR2), and through intracellular signaling to induce an anti-apoptotic effect as well as to enhance tumor growth, migration and angiogenesis. In addition, TF:FVIIa more indirectly facilitates metastatic dissemination through thrombin generation and PAR1 signaling (1-4).

Clinically, TF is overexpressed in a number of cancers including glioma, breast, colorectal, prostate, and pancreatic cancer (5-8). Within breast, gastric, esophageal, liver, colorectal and pancreatic cancer it has been shown that TF, measured by immunohistochemistry, is associated with increased metastatic disease and is a prognostic marker of poor overall survival (1).

Targeting TF has proven effective as a cancer therapy in preclinical models. Yu et al. demonstrated that silencing of TF by siRNA reduced tumor growth in a mouse model of colorectal cancer (11). Using an immunoconjugate with FVII as the binding domain, Hu et al. suppressed tumor growth in a human melanoma xenograft mouse model (12). Ngo et al. and Versteeg et al. demonstrated that anti-TF antibodies inhibited metastasis in an experimental metastasis model and suppressed tumor growth in a breast cancer model (13,14).

Targeting of TF with an antibody-drug conjugate (ADC) was recently shown to have a potent and encouraging therapeutic effect in murine cancer models, including patient-derived xenografts models (15). A non-invasive method for specific assessment of tumor TF expression status would be valuable. Such a tool would be clinically relevant for guidance of patient management and as companion diagnostics for emerging TF-targeting therapies.

Normally, TF is constitutively expressed on the surface of many extravascular cell types that are not in contact with the blood, such as fibroblasts, pericytes, smooth muscle cells and epithelial cells, but not on the surface of cells that come in contact with blood, such as endothelial cells and monocytes. However, TF is also expressed in various pathophysiological conditions where it is believed to be involved in progression of disease states within cancer, inflammation, atherosclerosis and ischemia/reperfusion. Thus, TF is now recognised as a target for therapeutic intervention in conditions associated with increased expression.

FVIIa is a two-chain, 50 kilodalton (kDa) vitamin-K dependent, plasma serine protease which participates in the complex regulation of in vivo haemostasis. FVIIa is generated from proteolysis of a single peptide bond from its single chain zymogen, Factor VII (FVII), which is present at approximately 0.5 μg/ml in plasma. The zymogen is catalytically inactive. The conversion of zymogen FVII into the activated two-chain molecule occurs by cleavage of an internal peptide bond. In the presence of calcium ions, FVIIa binds with high affinity to exposed TF, which acts as a cofactor for FVIIa, enhancing the proteolytic activation of its substrates FVII, Factor IX and FX.

In addition to its established role as an initiator of the coagulation process, TF was recently shown to function as a mediator of intracellular activities either by interactions of the cytoplasmic domain of TF with the cytoskeleton or by supporting the FVIIa-protease dependent signaling. Such activities may be responsible, at least partly, for the implicated role of TF in tumor development, metastasis and angiogenesis. Cellular exposure of TF activity is advantageous in a crisis of vascular damage but may be fatal when exposure is sustained as it is in these various diseased states. Thus, it is critical to regulate the expression of TF function in maintaining the health.

Radiolabeled TF agonists and/or TF antagonists may be valuable for diagnostic imaging with a gamma camera, a PET camera or a PET/CT camera, in particular for the evaluation of TF expression of tumor cells, for grading the malignancy of tumor cells known to express TF receptors, for the monitoring of tumors with TF expression during conventional chemotherapy or radiation therapy. Also TF agonists and/or TF antagonists labelled with alpha- or beta-emitting isotopes could be used for therapy, possibly with bi-specific binding to compounds with chemotherapeutic action, which may be related to the presence of TF receptors. In those cases the diagnostic imaging may be important for the evaluation of tumor response expected after therapy with TF receptor binding drugs.

Also other kinds of diseases with increased expression of surface accessible TF receptors may be observed, maybe inflammatory or auto-immune diseases, where both diagnostic and therapeutic application of radiolabeled TF agonists and/or TF antagonists may become relevant.

One example of a TF antagonist, inactivated FVII (FVIIai) is FVIIa modified in such a way that it is catalytically inactive. Thus, FVIIai is not able to catalyze the conversion of FX to FXa, or FIX to FIXa but still able to bind tightly to TF in competition with active endogenous FVIIa and thereby inhibit the TF function.

International patent applications WO 92/15686, WO 94/27631, WO 96/12800, WO 97/47651 relates to FVIIai and the uses thereof. International patent applications WO 90/03390, WO 95/00541, WO 96/18653, and European Patent EP 500800 describes peptides derived from FVIIa having TF/FVIIa antagonist activity. International patent application WO 01/21661 relates to bivalent inhibitor of FVII and FXa.

Hu Z and Garen A (2001) Proc. Natl. Acad. Sci. USA 98; 12180-12185, Hu Z and Garen A (2000) Proc. Natl. Acad. Sci. USA 97; 9221-9225, Hu Z and Garen A (1999) Proc. Natl. Acad. Sci. USA 96; 8161-8166, and International patent application WO 0102439 relates to immunoconjugates which comprises the Fc region of a human IgG1 immunoglobulin and a mutant FVII polypeptide, that binds to TF but do not initiate blood clotting.

Furthermore, International patent application WO 98/03632 describes bivalent agonists having affinity for one or more G-coupled receptors, and Burgess, L. E. et al., Proc. Natl. Acad. Sci. USA 96, 8348-8352 (July 1999) describes “Potent selective non-peptidic inhibitors of human lung tryptase”.

NIELSEN C. H. et al.(THE JOURNAL OF NUCLEAR MEDICINE, 2016, Vol 57, no. 1, pages 89-95) discloses the use of the imaging agent 18F-labeled human active site-inhibited factor VII in Positron Emission Tomography (PET) for identification and quantification of tissue factor expressing cancers in mice. The authors conclude in this paper that while 18F-FVIIai is a promising PET tracer for specific and noninvasive imaging of tumor TF expression the tracer merits further development and clinical translation, with potential to become a companion diagnostics for emerging TF targeted therapies.

There is still a need in the art for improved compounds, which efficiently inhibits pathophysiological TF function at relatively low doses and which does not produce undesirable side effects.

### SUMMARY OF THE INVENTION

The present inventors have surprisingly found that 18-F labelled factor VIIai (Egtl 18F-ASIS) is very useful for PET imaging of a Tissue Factor expressing tumor in a human. So far 18-F labelled human factor VIIa has only been validated in mice implanted with human xenografts (cf above mentioned paper). Although the human factor VIIa binds to such xenografts the mouse model is not a representative model for human use, since human factor VIIa does hardly bind to the murine TF. Accordingly, background signals from normal murine tissue is very low in such an animal model rendering any conclusion as to the applicability in humans highly speculative. In order to address the true applicability of 18-F labelled factor VIIa for PET scanning of tumors the present inventors have demonstrated in a murine and dog model (which is representative for human use since the used ligand binds strongly to canine TF) that the background signal surprisingly is sufficiently low for obtaining PET images useful for diagnostic purposes.

Specifically, the present invention provides a positron-emitting 18-F labelled factor VIIa (FVIIa) imaging agent for use in a diagnosis by PET imaging of a Tissue Factor expressing tumor in a human, said agent comprising native human FVIIa or a variant thereof radiolabeled with 18-F, wherein the agent is to be administered in a dose of 50-400 MBq followed by PET scanning 1-6 hours after the agent has been administered, quantification through SUVmax and/or SUVmean for thereby obtaining a PET image for the diagnosis of tumor tissue factor status for use of diagnosis, treatment monitoring of as a companion diagnostics based on target-to-background ratio or absolute uptake (SUV).

Preferably FVIIa is active site inhibited factor VIIa (FVIIai) of SEQ ID NO: 1. In a particularly preferred embodiment FVIIai has been labelled with N-succinimidyl 4-18F fluorobenzoate (18F-SFB) to produce:

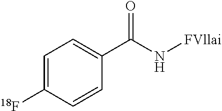

As elaborated on in the experimental part of the present disclosure the imaging agent of the invention is particularly useful in diagnosing breast, gastric, esophageal, liver, colorectal and pancreatic cancer.

In a further aspect the present invention provides a method for generating images of tissue factor expression in a human by diagnostic imaging involving administering the imaging agent of the invention to the human, and generating an image of at least a part of said body to which said imaging agent is administered.

The present invention is therefore also directed to a method of diagnosing by PET imaging a Tissue Factor expressing tumor in a human, said method comprising: administering a positron-emitting 18-F labelled factor VIIa (FVIIa) imaging agent, said agent comprising native human FVIIa or a variant thereof radiolabeled with 18-F, wherein the agent is administered in a dose of 50-400 MBq followed by PET scanning 1-6 hours after the agent has been administered, quantification through SUVmax and/or SUVmean for obtaining a PET image for the diagnosis of tumor tissue factor status.

Preferably, the method further includes the step of treatment monitoring of as a companion diagnostics based on target-to-background ratio or absolute uptake (SUV).

### BRIEF DESCRIPTION OF THE DRAWINGS

FIG. 1 shows PET images of mice with subcutaneous human pancreatic xenograft tumors detected with murine ¹⁸F-FVIIai (A) and human ¹⁸F-FVIIai (B).

FIG. 2 shows ex vivo bio-distribution of mice with BxPC-3 tumors performed 4 hours after injection of 18F-FVIIai of human or murine origin.

FIG. 3 shows human Factor VII having the sequence of SEQ ID NO: 1.

### DETAILED DESCRIPTION OF THE INVENTION

The 18-F labelled factor VIIa imaging agents of the present invention may be synthesized and purified in accordance with international patent application WO2004064870.

The terms “variant” or “variants”, as used herein, is intended to designate human Factor VII having the sequence of SEQ ID NO: 1, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. In one embodiment of the invention the variant has a total amont of amino acid substitutions and/or additions and/or deletions independently selected from the group consisting of 1, 2, 3, 4, 5, 6, 7, 8, 9, and 10. The activation of factor VII to factor VIIa, involves the hydrolysis of a single peptide bond between Arg152 and 11e153, resulting in a two-chain molecule consisting of a light chain of 152 amino acid residues and a heavy chain of 254 amino acid residues held together by a single disulfide bond.

Preferably, the FVIIa of SEQ ID NO: 1 is active site inhibited factor VIIa (FVIIai) and modified in such a way that it is catalytically inactive, such as having the amino acid modification comprised of Ser344, Asp242, and His193.

By “catalytically inactivated in the active site of the FVIIa polypeptide” is meant that a FVIIa inhibitor is bound to the FVIIa polypeptide and decreases or prevents the FVIIa-catalysed conversion of FX to FXa. A FVIIa inhibitor may be identified as a substance, which reduces the amidolytic activity by at least 50% at a concentration of the substance at 400 μM in the FVIIa amidolytic assay described by Persson et al. (Persson et al., J. Biol. Chem. 272: 19919-19924 (1997)). Preferred are substances reducing the amidolytic activity by at least 50% at a concentration of the substance at 300 μM; more preferred are substances reducing the amidolytic activity by at least 50% at a concentration of the substance at 200 μM.

The “FVIIa inhibitor” may be selected from any one of several groups of FVIIa directed inhibitors. Such inhibitors are broadly categorised for the purpose of the present invention into i) inhibitors which reversibly bind to FVIIa and are cleavable by FVIIa, ii) inhibitors which reversibly bind to FVIIa but cannot be cleaved, and iii) inhibitors which irreversibly bind to FVIIa. For a review of inhibitors of serine proteases see Proteinase Inhibitors (Research Monographs in cell and Tissue Physiology; v. 12) Elsevier Science Publishing Co., Inc., New York (1990).

The FVIIa inhibitor moiety may also be an irreversible FVIIa serine protease inhibitor. Such irreversible active site inhibitors generally form covalent bonds with the protease active site. Such irreversible inhibitors include, but are not limited to, general serine protease inhibitors such as peptide chloromethylketones (see, Williams et al., J. Biol. Chem. 264:7536-7540 (1989)) or peptidyl cloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosyllysylchloromethyl ketone (TLCK); nitrophenyl-sulphonates and related compounds; heterocyclic protease inhibitors such as isocoumarines, and coumarins.

### EXAMPLE 1

The in vivo properties of 18F-FVIIai for PET imaging was evaluated in a mouse model of human pancreatic cancer using small animal PET/CT. The uptake of 18F-FVIIai measured by PET was correlated with TF expression measured ex vivo to confirm specific imaging of tumor TF expression.

Active site inhibited factor VIIa (FVIIai) was obtained by inactivation with phenylalanine-phenylalanine-arginine-chloromethyl ketone. FVIIai was radiolabeled with N-succinimidyl 4-18Ffluorobenzoate (18F-SFB) and purified. The corresponding product, 18F-FVIIai, was injected into nude mice with subcutaneous human pancreatic xenograft tumors (BxPC-3) and investigated using small animal PET/CT imaging 1, 2 and 4 hours after injection. Ex vivo biodistribution was performed after the last imaging session, and tumor tissue was preserved for molecular analysis. A blocking experiment was performed in a second set of mice. The expression pattern of TF in the tumors was visualized by immunohistochemistry and the amount of TF in tumor homogenates was measured by ELISA and correlated with the uptake of 18F-FVIIai in the tumors measured in vivo by PET imaging.

The PET images showed high uptake of 18F-FVIIai in the tumor regions with a mean uptake of 2.5±0.3 percentage injected dose per gram (% ID/g) (Mean±SEM) 4 hours after injection of 7.3-9.3 MBq 18F-FVIIai and with an average maximum uptake in the tumors of 7.1±0.7% ID/g at 4 hours. In comparison, the muscle uptake was 0.2±0.01% ID/g at 4 hours. At 4 hours the tumors had the highest uptake of any organ. Blocking with FVIIai significantly reduced the uptake of 18F-FVIIai from 2.9±0.1 to 1.4±0.1% ID/g (P<0.001). The uptake of [18F]FVIIai measured in vivo by PET imaging correlated (r=0.72, P<0.02) with TF protein level measured ex vivo.

A limitation of the study is the absence of human TF outside the tumor regions in the xenograft mouse model applied. It has previously been shown that the cross species compatibility for TF/FVII is rather low such that human FVIIai binds with much lower affinity to murine than to human TF (28,33). Hence, the mouse model underestimates the background uptake of FVIIai to be seen in human tissues.

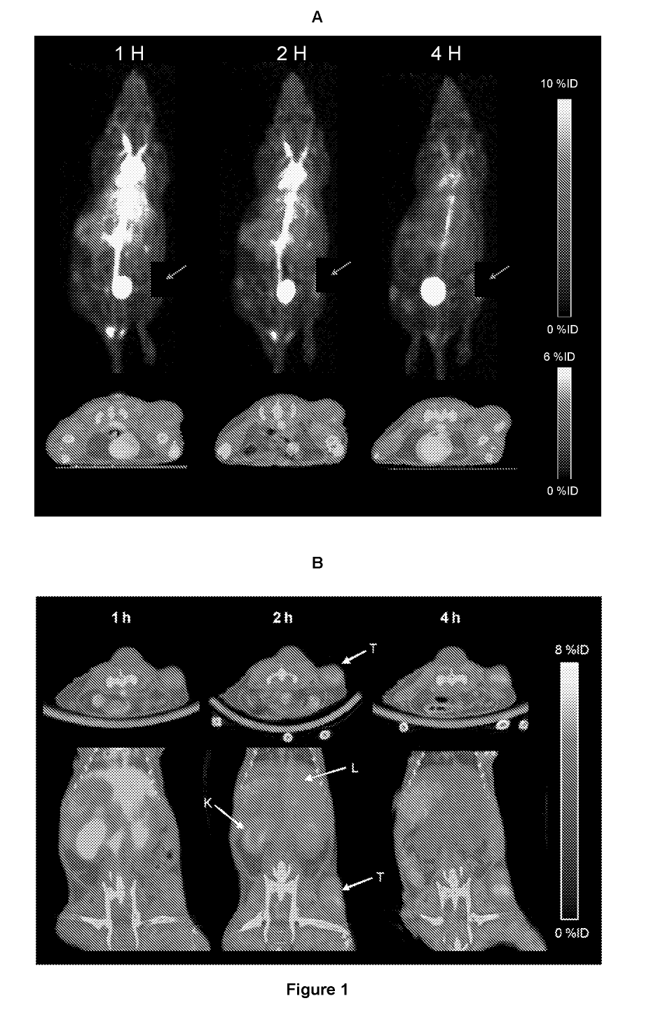

Referring to FIG. 1A there is shown mice with tissue factor positive human xenograft tumors (BxPC-3) were injected with murine ¹⁸F-FVIIai and scanned with a dedicated small animal PET/CT scanner at 1, 2 and 4 hours after injection. 18F-FVIIai accumulates in the tissue factor positive tumor (arrows) despite the endogenous factor VII. Also, and surprisingly the background expression of tissue factor in the animal does not impede the visualization of the tissue factor positive tumor. In FIG. 1B there is shown a corresponding experiment with 18F-FVIIai of human origin. The tumors are clearly visible (arrow) . The muscle uptake is still low and a good tumor to muscle contrast is obtained at 4 hours.

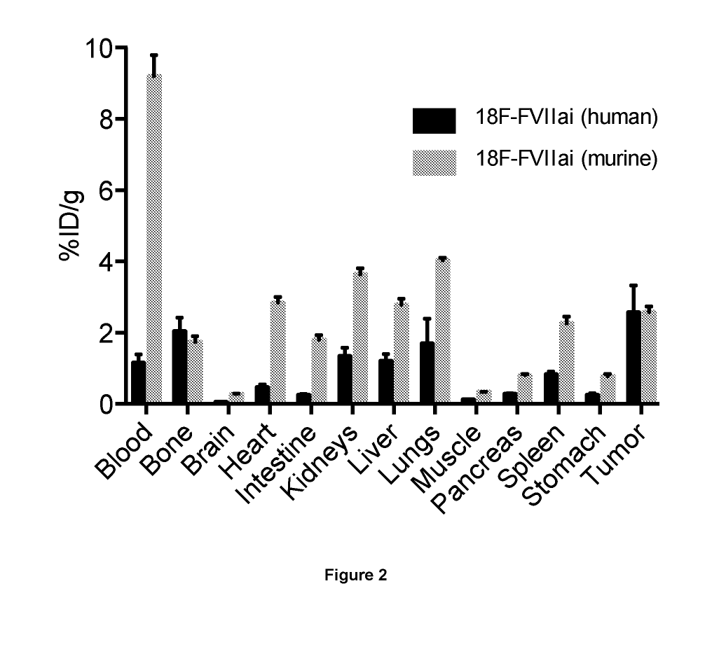

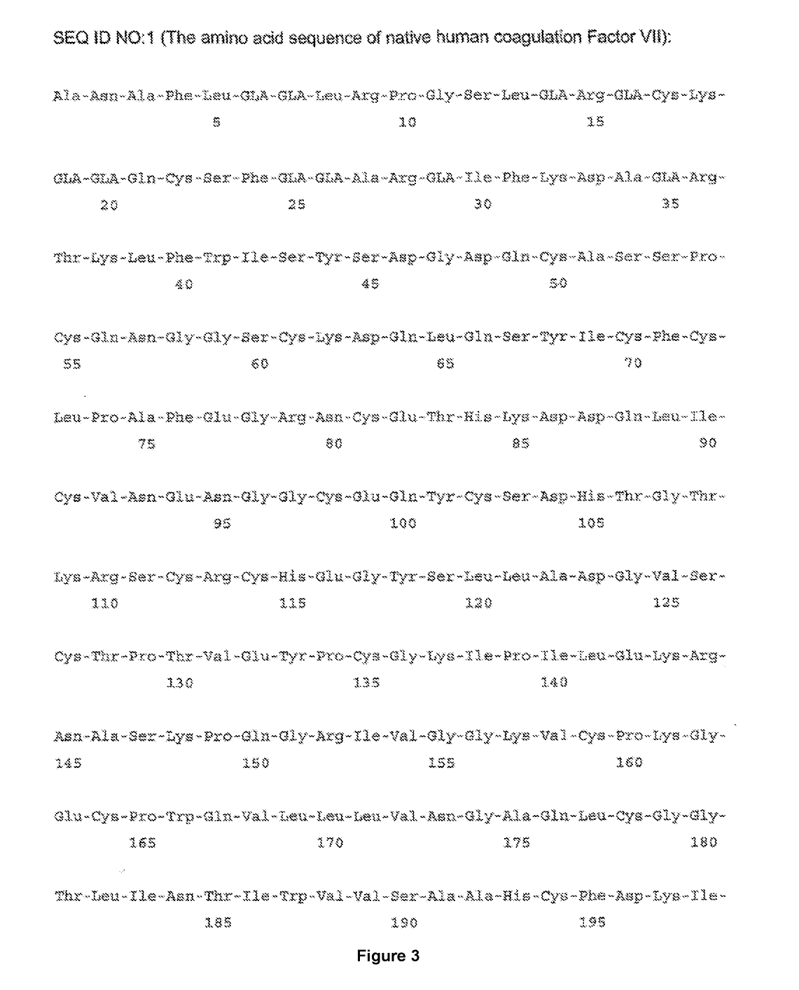

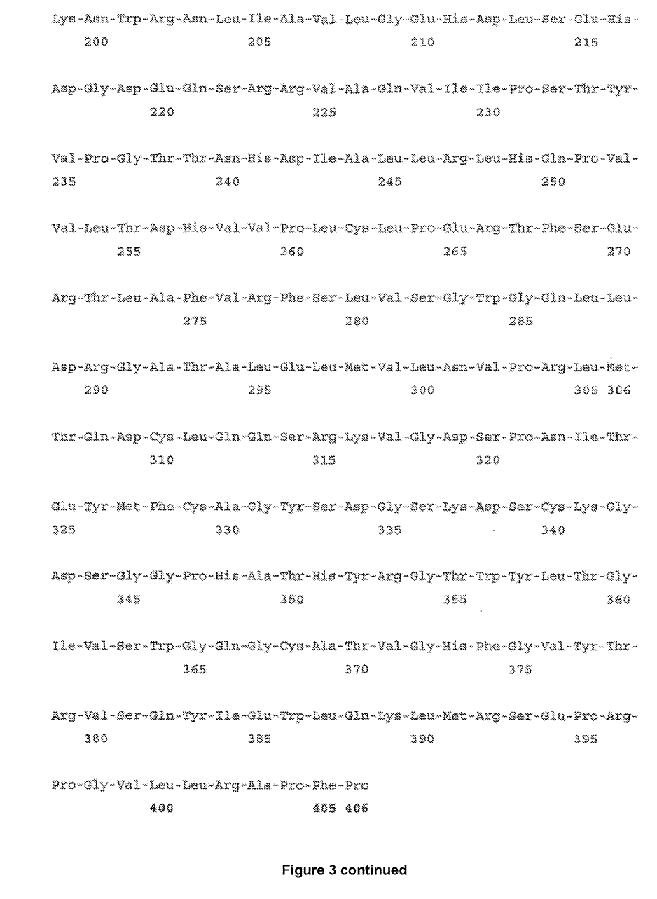

Referring to FIG. 2 there is shown ex vivo biodistribution of mice with BxPC-3 tumors performed 4 hours after injection of 18F-FVIIai of human or murine origin. The uptake of murine 18F-FVIIai in the tumors is not affected by the ability to bind murine tissue factor expressed in normal tissue or by competition with endogenous factor VII. The results from the ex vivo biodistribution clearly demonstrate that the biodistribution using human 18F-FVIIai that does not bind to mouse tissue factor can in no way predict the bio-distribution in the presence of background binding outside the tumor (murine tissue factor).

Surprisingly, the radiolabeled protein of the present invention is very useful in the visualization of TF positive tumors despite the presence of TF background expression and competition with endogenous FVII. This result merits the clinical translation.

### EXAMPLE 2

The in vivo properties of 18F-FVIIai for PET imaging was evaluated in a series of dogs with various spontaneous tumors using a clinical PET/CT scanner. The human 18F-FVIIai has high binding activity also to canine TF and demonstrated very useful in the visualization of TF positive tumors despite the presence of TF background expression and competition with endogenous FVII.

### REFERENCES

1. van den Berg Y W, Osanto S, Reitsma P H, Versteeg H H. The relationship between tissue factor and cancer progression: insights from bench and bedside. Blood. 2012; 119:924-32.
2. Ruf W, Yokota N, Schaffner F. Tissue factor in cancer progression and angiogenesis. Thromb Res. 2010; 125 Suppl 2:S36-8.
3. Kasthuri R S, Taubman M B, Mackman N. Role of tissue factor in cancer. J Clin Oncol. 2009; 27:4834-8.
4. Ruf W, Mueller B M. Thrombin generation and the pathogenesis of cancer. Semin Thromb Hemost. 2006; 32 Suppl 1:61-8.
5. Ueno T, Toi M, Koike M, Nakamura S, Tominaga T. Tissue factor expression in breast cancer tissues: its correlation with prognosis and plasma concentration. Br J Cancer. 2000; 83:164-70.
6. Seto S, Onodera H, Kaido T, et al. Tissue factor expression in human colorectal carcinoma: correlation with hepatic metastasis and impact on prognosis. Cancer. 2000; 88:295-301.
7. Khorana A A, Ahrendt S A, Ryan C K, et al. Tissue factor expression, angiogenesis, and thrombosis in pancreatic cancer. Clin Cancer Res. 2007; 13:2870-5.
8. Förster Y, Meye A, Albrecht S, Schwenzer B. Tissue factor and tumor: clinical and laboratory aspects. Clin Chim Acta. 2006; 364:12-21.
9. Nitori N, Ino Y, Nakanishi Y, et al. Prognostic significance of tissue factor in pancreatic ductal adenocarcinoma. Clin Cancer Res. 2005; 11:2531-9.
10. Kakkar A K, Lemoine N R, Scully M F, Tebbutt S, Williamson R C. Tissue factor expression correlates with histological grade in human pancreatic cancer. Br J Surg. 1995; 82:1101-4.
11. Yu J L, May L, Lhotak V, et al. Oncogenic events regulate tissue factor expression in colorectal cancer cells: implications for tumor progression and angiogenesis. Blood. 2005; 105:1734-41.
12. Hu Z, Sun Y, Garen A. Targeting tumor vasculature endothelial cells and tumor cells for immunotherapy of human melanoma in a mouse xenograft model. Proc Natl Acad Sci USA. 1999; 96:8161-6.
13. Ngo C V, Picha K, McCabe F, et al. CNTO 859, a humanized antitissue factor monoclonal antibody, is a potent inhibitor of breast cancer metastasis and tumor growth in xenograft models. Int J Cancer. 2007; 120:1261-7.
14. Versteeg H H, Schaffner F, Kerver M, et al. Inhibition of tissue factor signaling suppresses tumor growth. Blood. 2008; 111:190-9.
15. Breij E C W, de Goeij B E C G, Verploegen S, et al. An antibody-drug conjugate that targets tissue factor exhibits potent therapeutic activity against a broad range of solid tumors. Cancer Res. 2014; 74:1214-26.
16. Sorensen B B, Persson E, Freskgård P O, et al. Incorporation of an active site inhibitor in factor VIIa alters the affinity for tissue factor. J Biol Chem. 1997; 272:11863-8.
17. Tang G, Tang X, Wang X. A facile automated synthesis of N-succinimidyl 4-[18F]fluorobenzoate ([18F]SFB) for 18F-labeled cell-penetrating peptide as PET tracer. J Label Compd Radiopharm. 2010; 53:543-7.
18. Erlandsson M, Nielsen C H, Jeppesen T E, et al. Synthesis and characterization of (18)F-labeled active site inhibited factor VII (ASIS). J Label Compd Radiopharm. 2015; 58:196-201.
19. Madsen J, Kristensen J B, Olsen O H, et al. Recombinant coagulation factor VIIa labelled with the fac-99 mTc(CO)3-core: synthesis and in vitro evaluation of a putative new radiopharmaceutical for imaging in acute bleeding lesion. J Label Compd Radiopharm. 2011; 54:214-9.
20. Persson E. Influence of the gamma-carboxyglutamic acid-rich domain and hydrophobic stack of factor VIIa on tissue factor binding. Haemostasis. 1996; 26 Suppl 1:31-4.
21. Nalla A, Buch I, Sigvardt M, Bodholdt R P, Kjaer A, Hesse B. (111)Indium labelling of recombinant activated coagulation factor VII: In vitro and preliminary in vivo studies in healthy rats. Int J Mol Imaging. 2012; 2012:464810.
22. Temma T, Ogawa Y, Kuge Y, et al. Tissue factor detection for selectively discriminating unstable plaques in an atherosclerotic rabbit model. J Nucl Med. 2010; 51:1979-86.
23. Hong H, Zhang Y, Nayak T R, et al. Immuno-PET of tissue factor in pancreatic cancer. J Nucl Med. 2012; 53:1748-54.
24. Shi S, Hong H, Orbay H, et al. ImmunoPET of tissue factor expression in triple-negative breast cancer with a radiolabeled antibody Fab fragment. Eur J Nucl Med Mol Imaging. 2015; 42:1295-303.
25. Viola-Villegas N T, Sevak K K, Carlin S D, et al. Noninvasive imaging of PSMA in prostate tumors with (89)Zr-labeled huJ591 engineered antibody fragments: the faster alternatives. Mol Pharmaceutics. 2014; 11:3965-73.
26. Cirillo P, Golino P, Ragni M, et al. Long-lasting antithrombotic effects of a single dose of human recombinant, active site blocked factor VII: insights into possible mechanism(s) of action. J Thromb Haemost. 2003; 1:992-8.
27. Erhardtsen E, Nilsson P, Johannessen M, Thomsen M S. Pharmacokinetics and safety of FFR-rFVIIa after single doses in healthy subjects. J Clin Pharmacol. 2001; 41:880-5.
28. Petersen L C, Newby P L, Branner S, et al. Characterization of recombinant murine factor VIIa and recombinant murine tissue factor: a human-murine species compatibility study. Thromb Res. 2005; 116:75-85.
29. Muller M, Flössel C, Haase M, et al. Cellular localization of tissue factor in human breast cancer cell lines. Virchows Arch, B, Cell Pathol. 1993; 64:265-9.
30. Saito Y, Hashimoto Y, Kuroda J-I, et al. The inhibition of pancreatic cancer invasion-metastasis cascade in both cellular signal and blood coagulation cascade of tissue factor by its neutralisation antibody. Eur J Cancer. 2011; 47:2230-9.
31. Niu G, Li Z, Xie J, Le Q-T, Chen X. PET of EGFR antibody distribution in head and neck squamous cell carcinoma models. J Nucl Med. 2009; 50:1116-23.
32. Beeby T L, Chasseaud L F, Taylor T, Thomsen M K. Distribution of the recombinant coagulation factor 1251-rFVIIa in rats. Thromb Haemost. 1993; 70:465-8.
33. Knudsen T, Olsen O H, Petersen LC. Tissue factor and factor VIIa cross-species compatibility. Front Biosci (Landmark Ed). 2011; 16:3196-215.
34. Jilma B, Marsik C, Mayr F, et al. Pharmacodynamics of active site inhibited factor VIIa in endotoxin-induced coagulation in humans. Clinical Pharmacology & Therapeutics. 2002; 72:403-10.
35. Vincent J-L, Artigas A, Petersen L C, Meyer C. A multicenter, randomized, double-blind, placebo-controlled, dose-escalation trial assessing safety and efficacy of active site inactivated recombinant factor VIIa in subjects with acute lung injury or acute respiratory distress syndrome. Crit Care Med. 2009; 37:1874-80.
36. Neumaier B, Mottaghy F M, Buck A K, et al. Short Communication: (18)F-immuno-PET: Determination of anti-CD66 biodistribution in a patient with high-risk leukemia. Cancer Biother Radiopharm. 2008; 23:819-24.

1. A positron-emitting 18-F labelled factor VIIa (FVIIa) imaging agent for use in a diagnosis by PET imaging of a Tissue Factor expressing tumor in a human, said agent comprising native human FVIIa or a variant thereof radiolabeled with 18-F, wherein the agent is to be administered in a dose of 50-400 MBq followed by PET scanning 1-6 hours after the agent has been administered, quantification through SUVmax and/or SUVmean for thereby obtaining a PET image for the diagnosis of tumor tissue factor status for use of diagnosis, treatment monitoring of as a companion diagnostics based on target-to-background ratio or absolute uptake (SUV).

2. The 18-F labelled factor VIIa (FVIIa) imaging agent for use according to claim 1, wherein the FVIIa is SEQ ID NO: 1 and active site inhibited factor VIIa (FVIIai) and modified in such a way that it is catalytically inactive, such as having the amino acid modification comprised of Ser344, Asp242, and His193.

3. The 18-F labelled factor VIIa (FVIIa) imaging agent for use according to claim 2, wherein FVIIai has been labelled with N-succinimidyl 4-¹⁸Ffluorobenzoate (18F-SFB) to produce:

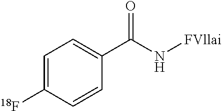

4. The 18-F labelled factor VIIa (FVIIa) imaging agent for use according to claim 1 to diagnose, stage or therapy monitoring in breast, gastric, esophageal, liver, colorectal and pancreatic cancer.

5. The 18-F labelled factor VIIa (FVIIa) imaging agent for use according to claim 1 as a diagnostic companion in a cancer entity where tissue factor directed therapy has been shown to be relevant, such as in breast, gastric, esophageal, liver, colorectal and pancreatic cancer.

6. T The 18-F labelled factor VIIa (FVIIa) imaging agent for use according to claim 1, wherein agent is to be administered in a dose of 50-400 MBq.

7. T A method for generating images of tissue factor expression in a human by diagnostic imaging involving administering the imaging agent of claim 1 to said human, and generating an image of at least a part of said body to which said imaging agent is administered.

8. A method of diagnosing by PET imaging a Tissue Factor expressing tumor in a human, said method comprising:
administering a positron-emitting 18-F labelled factor VIIa (FVIIa) imaging agent, said agent comprising native human FVIIa or a variant thereof radiolabeled with 18-F,
wherein the agent is administered in a dose of 50-400 MBq followed by PET scanning 1-6 hours after the agent has been administered, quantification through SUVmax and/or SUVmean for obtaining a PET image for the diagnosis of tumor tissue factor status.

9. Method according to claim 8, further including the step of treatment monitoring of as a companion diagnostics based on target-to-background ratio or absolute uptake (SUV).

10. Method according to claim 8 using the FVIIa of SEQ ID NO: 1, which is active site inhibited factor VIIa (FVIIai) and modified in such a way that it is catalytically inactive, such as having the amino acid modification comprised of Ser344, Asp242, and His193.

11. Method according to claim 10, wherein FVIIai has been labelled with N-succinimidyl 4-18Ffluorobenzoate (18F-SFB) to produce:

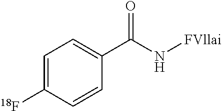

12. Method according to claim 8 to diagnose, stage or therapy monitoring in breast, gastric, esophageal, liver, colorectal and pancreatic cancer
